(19) United States
(12) Patent Application Publication
(10) Pub. No.: US 2020/0009033 A1
(43) Pub. Date: Jan. 9, 2020

(54) ORAL CARE FORMULATIONS INCORPORATING TETRAHYDROCURCUMINOIDS AND METHODS FOR PREPARING SAME

(71) Applicant: Vingt Holdings, LLC, New York, NY (US)

(72) Inventor: Michael Gulizio, New York, NY (US)

(73) Assignee: Vingt Holdings, LLC, New York, NY (US)

(21) Appl. No.: 16/027,853

(22) Filed: Jul. 5, 2018

(51) Int. Cl. A61K 8/35; A61Q 11/00; A61K 8/9789

(52) U.S. Cl. CPC A61K 8/35; A61K 2800/92; A61K 8/9789; A61Q 11/00

(57) ABSTRACT

The present invention provides oral care compositions comprising one or more tetrahydrocurcumin compounds. It has unexpectedly been found that the addition of specific tetrahydrocurcuminoids in a specific way not only improves incorporation of the compounds in oral care formulations, but further yields formulations exhibiting increased bioavailability and decreased oxidation in comparison to previously available curcumin-containing oral care compositions.

### BACKGROUND OF THE INVENTION

Periodontal disease (“gum disease”) is a broad term that refers to diseases attacking the gingiva and the underlying alveolar bone supporting the teeth. The term is used for any inflammatory disease which initially occurs at a marginal gingiva area and may affect the alveolar bone. Periodontal disease can affect the periodontium, as well as the supporting tissue surrounding a tooth (i.e., the periodontal ligament, the gingiva, and the alveolar bone). Two common stages of periodontal disease are gingivitis (inflammation of the gingiva) and periodontitis (inflammation of the periodontal ligament resulting in progressive resorption of alveolar bone, increasing mobility of the teeth, and loss of the teeth at advanced stages). Combinations of inflammatory and degenerative conditions are termed periodontitis complex.

Periodontal disease most often leads to one or more of the following conditions: inflammation of the gingiva, formation of periodontal pockets and bleeding and/or pus discharge from these pockets, resorption of alveolar bone, loose teeth and loss of teeth. Bacteria present in dental plaque which forms on the surface of the teeth and in the periodontal pocket contributes to both the initiation and progress of periodontal disease. In order to prevent or treat periodontal disease, these bacteria must be suppressed by means other than simple mechanical scrubbing. Significant research has been conducted towards developing therapeutic dentifrices and mouthwashes for treating periodontal disease by suppressing these bacteria. However, periodontal disease involves more than the bacterial infection. Severe periodontal disease involves the destruction of periodontal tissue, which is primarily caused by the inflammation resulting from the host's reaction to the bacteria in the periodontium and gingival sulcus.

Early stage gingivitis is an inflammatory disease of the gingival and periodontium characterized by redness, swelling and bleeding upon probing. Anaerobic bacteria are generally regarded as the initiating agent of gingivitis, with subsequent progression and disease severity determined by the host immune response, i.e., inflammation, which is a nonspecific cellular and biochemical process involving multiple pro-inflammatory agents.

Left untreated, gingivitis usually results in the formation of periodontal pockets, with the bacteria in the plaque attacking the periodontium (periodontal tissues supporting the teeth), causing a chronic inflammation of the periodontium known as periodontitis. In advanced stages of periodontitis, the bacteria eventually erode the bone surrounding the teeth, often resulting in attachment loss, bone destruction and loss of the affected teeth. Current therapies for gingivitis and/or periodontitis include improved oral hygiene to eliminate supragingival plaque and calculus, professional prophylaxis to eliminate subgingival calculus, and/or surgery.

Recent research further indicates that periodontal disease may be a far more serious threat to overall systemic health than previously realized. For example, the presence of periodontitis can also result in the release of bacteria and/or bacterial toxins into the bloodstream. Host responses to the presence of these bacterial pathogens and/or toxins in the bloodstream may contribute to the development of atherosclerosis (heart disease) and pose a serious threat to people whose health is compromised by diabetes, severe respiratory diseases, stroke and bacteremia (bacteria in the blood).

It now appears that people with periodontitis may be more at risk for heart disease, and have a significantly higher risk for fatal heart attacks, than patients without periodontitis. Heart disease is the leading cause of death in most developed countries, and periodontitis is one of the most common bacterial-mediated diseases in humans, affecting as many as one third of those over 50. Even if periodontitis has only a modest effect on increasing the risk of heart attack, its prevalence may make it a significant contributor to the risk for heart disease in the population as a whole.

Several theories attempt to explain the link between heart disease and periodontal disease. Researchers have found that 70% of the fatty plaque that blocks carotid arteries and causes stroke contains bacteria, and 40% of those bacteria have been traced to the mouth. One theory is that oral bacterial pathogens enter the blood through inflamed gums, attach to fatty plaques in the coronary arteries (heart blood vessels) and cause small blood clots. Coronary artery disease is characterized by a thickening of the walls of the coronary arteries due to the buildup of fatty plaque. Blood clots further obstruct normal blood flow, restricting the amount of nutrients and oxygen required for the heart to function properly. This may lead to heart attacks. Another theory is that changes in systemic inflammatory mediators caused by periodontitis increase development of atherosclerotic plaque, which then contributes to thickening of the arterial walls.

Research also suggests that people with diabetes are more likely to have periodontitis than people without diabetes, and the presence of periodontitis may make it more difficult for diabetics to control their blood sugar. The presence of periodontitis can increase blood sugar, contributing to increased periods of time when the body functions with a high blood sugar level, which puts a diabetic person at increased risk for diabetic complications. Thus, controlling periodontitis may also help control diabetes. One study (“Heightened Gingival Inflammation and Attachment Loss in Type 2 Diabetics with Hyperlipidemia,” in the Journal of Periodontology, November, 1999) found that poorly controlled type 2 diabetic patients are more likely to develop periodontal disease than well-controlled diabetics. The study further explains why diabetics are more susceptible to severe periodontal disease. Poorly controlled diabetics respond differently to bacterial plaque at the gum line than well-controlled diabetics and non-diabetics, possibly due to elevated serum triglycerides. Poorly controlled diabetics have more harmful proteins (cytokines) in their gingival tissue, causing destructive inflammation of the gums. In turn, beneficial proteins (growth factors) are reduced, interfering with the healing response to infection.

Periodontal disease may also pose an increased risk for severe respiratory diseases like pneumonia, bronchitis, emphysema and chronic obstructive pulmonary disease. The VA Dental Longitudinal Study (DLS) and Normative Aging Study (NAS) examined the relationship of periodontal disease to mortality from all outcomes and concluded that periodontal status at baseline was a significant and independent predictor of mortality. [Annals of Periodontology, 3(1), 339-49, July 1998] The study started in the mid-1960s enrolling men in good medical health and tracked them for more than 25 years. It was found that for each 20% increment in mean whole-mouth ABL (alveolar bone loss, measured with a Schei ruler using full-mouth series of periapical films), the subject's risk of death increased by 51%. The risk of death was also found to be associated with periodontal status as measured clinically by periodontal probing depth. Subjects in the population group with the deepest average probing depths were found to be at 74% higher risk.

The present invention relates to oral compositions for the prevention and treatment of dental caries, periodontal diseases such as gingivitis and periodontitis, other oral diseases, and overall systemic health, comprising at least one tetrahydrocurcuminoid, its derivatives, and analogues, from the plant Curcuma longa or other curcumin-containing plants and a safe and effective amount of at least one antioxidant such as an essential oil. The present invention also relates to methods for preparing said compositions.

### SUMMARY OF THE INVENTION

Curcumin (diferuloylmethane) is a naturally occurring compound obtained from the rhizomes of Curcuma longa plants. It is a major component of turmeric and commonly used as a spice (curry) and coloring agent for foods, drugs, and cosmetics.

Curcumin compounds have antioxidant properties and may be used in oral care compositions to exert beneficial physiological effects, such as prevention or amelioration of gingivitis or periodontitis. SU 1,132,945 discloses incorporating extracts of turmeric or ginger into toothpaste compositions for improved anti-inflammatory effect on tissues of the oral cavity and treatment of certain diseases of the mucous membranes of the oral cavity and marginal periodontitis. GB 2317339 discloses oral compositions for prevention and treatment of dental caries, periodontal diseases and other diseases of the oral cavity. The compositions include a curcuminoid and a fluoride ion source, in combination with oral care active ingredients and carrier materials.

However, attempts to incorporate curcumin compounds into oral care compositions (such as toothpastes) have met with a number of problems. First, curcumin does not easily mix into oral care compositions. Second, degradation and discoloration have been observed on storage, especially at elevated temperatures. Third, and most importantly, the bioavailability of curcumin in such compositions has been limited.

The present invention overcomes the drawbacks associated with previous curcumin products by providing oral care compositions comprising one or more tetrahydrocurcumin compounds. The present invention further provides novel oral care compositions and methods for producing such compositions. It has unexpectedly been discovered that pre-blending the tetrahydocurcumin compounds with particular essential oils significantly improves their incorporation in the finished product.

More specifically, the oral compositions of the present invention comprise: a) from about 0.001% to about 10% by weight, of a tetrahydrocurcuminoid such as tetrahydrocurcumin, its derivatives, analogues, and mixtures thereof, and b) from about 0.005% to about 10% of an essential oil, or mixtures of essential oils. Most preferably, the tetrahydrocurcuminoid is a 99% pure form of 1,7-Bis(4-hydroxy-3-methoxyphenyl)-3,5-heptanedione.

### DETAILED DESCRIPTION OF THE INVENTION

Curcumin is a polyphenolic yellow pigment which occurs naturally in the rhizome of the species Curcuma longa, and is the main active ingredient in turmeric. Curcumin is also known as diferuloylmethane or (1E,6E)-1,7-bis(4-hydroxy-3-methoxy-phenyl)hepta-1,6-diene-3,5-dione. Turmeric contains curcumin along with other natural analogues of curcumin which are collectively called “curcuminoids”. The major curcuminoids present in turmeric are demethoxycurcumin (also known as p-hydroxycinnamoyl(feruloyl)methane), bis-demethoxycurcumin (also known as p,p′-dihydroxydicinnamoylmethane) and cyclocurcumin (in which the alpha, beta-unsaturated beta-diketone group of curcumin is replaced by an alpha, beta-unsaturated dihydropyranone group). The term “curcumin compound” for the purposes of the present invention, encompasses curcumin, the curcuminoids, other natural or synthetic derivatives or analogues of curcumin, and also preparations of the plant Curcuma longa or other curcumin-containing plants, such as Curcuma xanthorrhiza, Curcuma zedoaria and Curcuma aromatica.

In the most preferred embodiment, the present invention relates to the use of a greater than 99% pure form of 1,7-Bis(4-hydroxy-3-methoxyphenyl)-3,5-heptanedione in the manufacture of oral care compositions. This highly purified form of 1,7-Bis(4-hydroxy-3-methoxyphenyl)-3,5-heptanedione shows enhanced activity and performance over other curcuminoids, including the corresponding 95% pure form, apparently because it is devoid of 5-Hydroxy-1,7-bis(4-hydroxy-3-methoxyphenyl)-3-heptanone or 3,5-Dihydroxy-1,7-bis(4-hydroxy-3-methoxyphenyl)-heptane.

Active and other useful ingredients may be categorized or described by their therapeutic and/or cosmetic benefit or their postulated mode of action or function. However, it is to be understood that the active and other ingredients useful herein can, in some instances, provide more than one cosmetic and/or therapeutic benefit or function synergistically or operate via more than one mode of action. Therefore, classifications herein are made for the sake of convenience and are not intended to limit an ingredient to the particularly stated application or applications listed.

The present compositions are used to treat and prevent diseases and conditions of the oral cavity including periodontal disease, thereby promoting and enhancing whole body or systemic health for the individual being treated. The oral care compositions of the invention may be in any form common in the art, including therapeutic rinses, especially mouth rinses; dentifrices such as toothpastes, tooth gels, and tooth powders; non-abrasive gels; mouth sprays; mousse; foams; chewing gums, lozenges and breath mints; dental solutions and irrigation fluids; and dental implements such as dental floss and tape.

One preferred product form is a dentifrice. The term “dentifrice” denotes paste, cream, mousse, aerosol, powder, gel, and/or liquid formulations which are used to clean the surfaces of the oral cavity. The dentifrice is an oral composition that is not intentionally swallowed for purposes of systemic administration of therapeutic agents, but is retained in the oral cavity for a sufficient time to contact substantially all of the dental surfaces and/or mucosal tissues for purposes of oral activity. Preferably the dentifrice is in the form of a paste or a gel (or a combination thereof).

A dentifrice composition according to the invention will generally contain further ingredients to enhance performance and/or consumer acceptability. In general, most toothpastes include such additional ingredients as: abrasives, binders, foaming ingredients, humectants, detergents, flavors, colors, preservatives, fluoride and water.

Water employed in the preparation of commercially suitable oral compositions should preferably be deionized and free of organic impurities. Water generally comprises from about 2% to about 99%, preferably from about 20% to about 95%, by weight of the dental compositions herein. When in the form of toothpastes, the compositions preferably are from about 2% to about 45%, more preferably from about 30% to about 40% water, while mouthwashes are preferably from about 45% to about 95%, more preferably from about 75% to about 90% water. These amounts of water include the free water which is added plus that which is introduced with other materials such as with sorbitol and other humectants (see below). The amount of humectant generally ranges from 0 to 70% by weight (based on the total weight of the dentifrice). Typical humectants include glycerol, sorbitol, polyethylene glycol, polypropylene glycol, propylene glycol, xylitol (and other edible polyhydric alcohols), hydrogenated partially hydrolyzed polysaccharides, aloe vera, and mixtures thereof.

Most toothpastes contain at least 50 percent abrasives. Baking soda, calcium carbonate, calcium phosphates, alumina, and silica are examples of abrasives. A dentifrice will usually comprise an abrasive cleaning agent in an amount of from about 3 to 75% by weight based on the total weight of the dentifrice. Suitable abrasive cleaning agents also include silica xerogels, hydrogels and aerogels and precipitated particulate silicas, calcium carbonate, dicalcium phosphate, tricalcium phosphate, calcined alumina, sodium and potassium metaphosphate, sodium and potassium pyrophosphates, sodium trimetaphosphate, sodium hexametaphosphate, particulate hydroxyapatite, dicalcium orthophosphate dihydrate, calcium pyrophosphate, tricalcium phosphate, calcium polymetaphosphate, insoluble sodium polymetaphosphate, hydrated alumina, and mixtures thereof. Silica dental abrasives of various types are preferred because of their unique benefits of exceptional dental cleaning and polishing performance without unduly abrading tooth enamel or dentin. The silica abrasive polishing materials useful herein, as well as other abrasives, generally have an average particle size ranging between about 0.1 and 30 microns, preferably between about 5 and 15 microns.

Fluorides are the most frequently used active ingredient in toothpaste. The three types of fluoride most commonly used are:
Sodium fluoride (NaF) is the type of fluoride used most often in toothpaste.
Stannous fluoride (SnF₂) is also used. This type may stain the tooth surface yellow or brown.
Monofluorophosphate (Na₂PO₃F).
   The use of fluoride, though common, remains controversial. Its many side effects arguably outweigh its benefits. While it can be included in toothpastes made in accordance with the present invention, the use of fluoride is not preferred.

Sodium lauryl sulfate (SLS) is frequently used as a surfactant. This is a detergent that also acts as a foaming agent. SLS is a harsh ingredient and is not preferred in the present invention. Other typical surfactants include anionic surfactants, such as the sodium, magnesium, ammonium or ethanolamine salts of C8 to C18 alkyl sulphates (including SLS), C8 to C18 alkyl sulphosuccinates (e.g., dioctyl sodium sulphosuccinate), C8 to C18 alkyl sulphoacetates (e.g., sodium lauryl sulphoacetate), C8 to C18 alkyl sarcosinates (e.g., sodium lauryl sarcosinate), C8 to C18 alkyl phosphates (optionally comprising up to 10 ethylene oxide and/or propylene oxide units) and sulphated monoglycerides. Other suitable surfactants include nonionic surfactants, such as optionally polyethoxylated fatty acid sorbitan esters, ethoxylated fatty acids, esters of polyethylene glycol, ethoxylates of fatty acid monoglycerides and diglycerides, ethylene oxide/propylene oxide block polymers, and amphoteric surfactants, such as betaines or sulphobetaines. Mixtures of any of the above described materials may also be used.

Antibacterial agents such as Triclosan are often included in toothpastes. Long-term use of any antibacterial ingredient can be problematic and the use of triclosan is not preferred in the present invention.

Flavoring agents can also be added to the present compositions. Examples of flavoring agents include oil of peppermint, oil of sassafras, clove bud oil, peppermint, menthol, anethole, thymol, methyl salicylate, eucalyptol, cassia, 1-menthyl acetate, sage, eugenol, parsley oil, oxanone, oil of wintergreen, alpha-irisone, oil of spearmint, marjoram, lemon, orange, propenyl guaethol, cinnamon, and mixtures thereof. These essential oils serve a dual purpose in the present invention. Flavoring agents are generally used in toothpastes at levels of from about 0.001% to about 5% by weight of the composition. Natural toothpastes often use more exotic flavors made from tea, aloe vera, anise, fennel, lavender, and other plants. Other preferred essential oils include goji berry and grape seed. In the present invention, essential oils play another critical role beyond flavoring. They also serve as a vehicle for dissolving the tetrahydracurcuminoids prior to incorporation with the remainder of the composition. Doing so results in a superior final product. It has unexpectedly been found that the essential oil/curcuminoids mix is more readily incorporated into the finished product, thereby increasing the bioavailability of the curcuminoids.

Some toothpastes are formulated to help strengthen enamel (outer surface of the tooth). Calcium phosphate is typically the remineralization ingredient used.

Humectants give toothpaste texture and help retain moisture. Suitable humectants include polyethylene glycol, sorbitol, glycerin, aloe vera, xylitol, other edible polyhydric alcohols, and mixtures thereof, at a level of from about 0% to about 70%, preferably from about 2% to about 55%, by weight of the compositions herein. Xylitol is particularly preferred since it has multiple benefits, including increasing the flow of saliva, which helps prevent dry mouth, and has also been shown in preliminary testing to help prevent tooth decay.

The dentifrice will usually contain a binder or thickening agent in an amount of from 0.5 to 10% by weight based on the total weight of the dentifrice. Suitable binders or thickening agents include carboxyvinyl polymers (such as polyacrylic acids cross-linked with polyallyl sucrose or polyallyl pentaerythritol), hydroxyethyl cellulose, hydroxypropyl cellulose, water soluble salts of cellulose ethers (such as sodium carboxymethyl cellulose and sodium carboxymethyl hydroxyethyl cellulose), natural gums (such as carrageenan, gum karaya, guar gum, xanthan gum, gum arabic, and gum tragacanth), finely divided silicas, hectorites, colloidal magnesium aluminium silicates and mixtures thereof. Xanthan gum is preferred.

To help prevent microorganisms from growing in toothpaste, certain preservatives are added. These include sodium benzoate, methyl paraben, and ethyl paraben.

Sweetening agents can be added to the present compositions. These include aspartame, acesulfame, sodium saccharin, dextrose, sucrose, lactose, maltose, stevia, xylitol, levulose, sodium cyclamate and mixtures thereof. Sweetening agents are generally used in toothpastes at levels of from about 0.005% to about 5% by weight of the composition. Stevia and xylitol are preferred.

Artificial dyes and coloring agents are not uncommon in most commercial toothpaste. Titanium dioxide is frequently used to make toothpaste white. Preferred embodiments of the present invention avoid artificial dyes and colorings.

The invention is further illustrated with reference to the following, non-limiting Examples. The examples further describe and demonstrate preferred embodiments within the scope of the present invention. The examples are given solely for illustration and are not to be construed as limitations of this invention as many variations are possible without departing from the spirit and scope thereof.

### EXAMPLE 1

### Toothpaste

| Ingredients | Wt. % |
|---|---|
| WATER (RO) | 30.3300 |
| DICALCIUM PHOSPHATE DIHYDRATE | 8.0000 |
| SODIUM BENZOATE EDF | 1.2500 |
| TIXOSIL 73 | 5.8000 |
| STEVIA 90% PE | 0.2500 |
| AROSIL 200 SILICA | 2.5000 |
| (XYLISORB 300) XYLITOL | 1.0000 |
| (NEOSORB P60W) SORBITOL POWDER | 5.0000 |
| ACTIPHTYE GOJI BERRY | 0.0100 |
| ALOE VERA GEL | 0.0100 |
| PERLASTAN L30 | 1.2000 |
| WINTER MINT OIL | 1.2000 |
| TETRAPURE TETRAHYDRACURCUMIN | 0.5000 |
| GRAPE SEED OIL DESERT WHALE | 0.0500 |
| POLYSORBATE 20 | 2.0000 |
| GLYCERINE 99.5% | 40.0000 |
| XANTHAN GUM | 0.9000 |

The ingredients can be mixed in any conventional manner. However, it has unexpectedly been determined that superior results may be obtained by pre-mixing the curcuminoids with one or more of the essential oils prior to incorporation with the remaining ingredients.
Another preferred formulation is a mouthwash:

### EXAMPLE 2

### Mouthwash

| Ingredients | Wt. % |
|---|---|
| WATER (RO) | 80.2790 |
| (NEOSORB P60W) SORBITOL POWDER | 2.0000 |
| (XYLISORB 300) XYLITOL | 2.0000 |
| SODIUM BENZOATE EDF | 1.0000 |
| STEVIA 90% PE | 0.2000 |
| ALOE VERA GEL | 0.0100 |
| GLYCERINE 99.5% | 4.0000 |
| T-MAZ 20 (POLYSORBATE 20) | 3.2000 |
| ACTIPHTYE GOJI BERRY | 0.0100 |
| TWEEN 80 (POLYSORBATE 80) | 3.2000 |
| WINTER MINT OIL | 1.0000 |
| GRAPE SEED OIL DESERT WHALE | 0.0010 |
| TETRAPURE TETRAHYDRACURUMIN | 0.1000 |
| PROPANEDIOL | 3.0000 |

Again, the ingredients can be mixed in any conventional manner. However, it has unexpectedly been determined that superior results may be obtained by pre-mixing the curcuminoids with one or more of the essential oils prior to incorporation with the remaining ingredients.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. An oral care composition comprising:
one or more tetrahydrocurcuminoids, each of said one or more tetrahydrocurcuminoids having a purity level of at least 99%;
one or more essential oils; and
a suitable carrier.

2. An oral care composition according to claim 1, in which the tetrahydrocurcuminoid is a 99% pure form of 1,7-Bis(4-hydroxy-3-methoxyphenyl)-3,5-heptanedione.

3. An oral care composition according to claim 1, wherein said composition is selected from the group comprising toothpastes, tooth gels, and tooth powders.

4. An oral care composition according to claim 1, wherein said essential oil is selected from the group comprising: oil of peppermint, oil of wintergreen, oil of spearmint, aloe vera, and mixtures thereof.

5. (canceled)

6. (canceled)

7. (canceled)

8. (canceled)
